(19) United States
(12) Patent Application Publication
(10) Pub. No.: US 2014/0038227 A1
(43) Pub. Date: Feb. 6, 2014

(54) APPARATUS AND METHOD FOR PREPARING CYTOLOGICAL SPECIMENS FOR ANALYSES

(71) Applicant: Peters Aremu, Orpington (GB)

(72) Inventor: Peters Aremu, Orpington (GB)

(21) Appl. No.: 14/110,158

(22) PCT Filed: Apr. 10, 2012
(86) PCT No.: PCT/GB2012/050785

§ 371 Date: Oct. 7, 2013

(30) Foreign Application Priority Data
Apr. 6, 2011 (GB) 1105789.0

(51) Int. Cl. C12Q 1/24

(52) U.S. Cl. CPC C12Q 1/24
USPC 435/30

(57) ABSTRACT

The apparatus (10) comprises receiving means for receiving at least one deposit of a cytological specimen, first and second staining stations (84, 86), and transfer means for transferring a portion of the or each deposit from the receiving means to the first staining station (84) and/or a portion of the or each deposit to the second staining station (86), the first staining station (84) being operable for a Papanicolaou staining technique and the second staining station (86) being operable for a Papanicolaou staining technique and at least one further staining technique. The method includes receiving a cytological specimen for pre-analysis preparation and adding anti-coagulant to the cytological specimen so as to improve the quality of information obtainable therefrom during later analysis.

The present invention relates to an apparatus and method for preparing pre-analysis cytological specimens, and more particularly but not necessarily exclusively to an apparatus and method for preparing gynaecological and non-gynaecological specimens.

Cytology is the study and analysis of the structure, formation, and function of cells. Healthy cells often display certain physical and physiological features that are characteristic of its specific cell type and which differentiate them from other cell types. These can be, for example, the shape and size of the cell, or its interaction with cells of the same type and of different types. By carrying out a visual examination of a specimen of a patient's cells, usually under a microscope, medical or health professionals are able to make diagnoses and evaluations of the patient's condition, based on any abnormalities observed. This is commonly known as diagnostic cytology.

Cytological samples can be obtained in a variety of techniques. A sample can be carried out by scraping or swabbing an area, such as the interior of a mouth. Fine needle aspiration can also be carried out by using a needle to aspirate material from palpable swelling of pathological suspicion and/or less accessible areas such as the lungs, liver, or bones.

Gynaecological samples, such as gynaecological cells obtained during a cervical smear test, are one of the most common types of cell studied in diagnostic cytology. Traditionally, a sample obtained from the cervix is smeared directly onto a microscope slide and then fixed with an alcohol based fixative before being stained with Papanicolaou stain. This is commonly known as a ‘Pap smear’, or a ‘Pap test’. The conventional screening method can sometimes produce inadequate smears, where an insufficient quantity of cells are transferred to the slide, and excessive blood and mucus on the slide makes diagnosis difficult.

In recent years, a different technique has been used increasingly, in which a sample of cells to be studied is placed into a vial containing liquid preservative. Once in the laboratory, the sample is agitated to achieve an even distribution of cells. The sample is then collected from the vial, any exudates, blood, or mucus removed by centrifugation or blended in a high-speed spin, and the cells spread out on a slide. The smeared cells are then fixed and stained ready for examination.

Non-gynaecological samples, such as urine, pleural fluid and ascetic fluid can also be Pap stained for microscopic examination. Other staining techniques are also used for non-gynaecological samples, such as the May-Grunwald staining technique, Periodic acid-Schiff staining, and Gram staining. The May-Grunwald technique, which is a peer staining technique to Pap testing, usually requires the specimens to be air dried and not fixed by an alcohol based fixative. The current practice of collecting a sample for such staining techniques is to place the sample in a blank container with no fixative or preservative. Without the May-Grunwald staining technique, non-gynaecological cell investigations are at risk of misinterpretation or less confident diagnoses.

Equipment is currently available that automates the staining of gynaecological samples. However, there is currently no equipment available that fully automates the processing of staining techniques other than Pap staining. Non-Pap staining techniques are conventionally carried out at least partially manually. However, the success of manual intervention depends on the experience level of the individual user. Inexperience can lead to inadequately prepared specimens being smeared and stained for analysis.

Furthermore, currently, for a sample that requires more than one type of staining technique, each staining technique needs to be processed separately. Sometimes this means the sample may need to be prepared by different machines or prepared manually making processing more tedious and inconsistent. This means that more than one sample may need to be collected in order to provide a sufficient quantity for processing using the different techniques and/or for sending to the different laboratories. This can increase the cost and opportunities for errors, not to mention the stress and discomfort a patient may experience with the collection of repeated samples if such preparations turn out to be inadequate.

Although there are automated processors available for preparing Pap stains, if the sample also requires other staining techniques, the non Pap stains will still need to be processed at least partially manually. Should the medical professional wish to examine all of the slides at the same time in order to gather a comprehensive overview of the health of the patient, he or she needs to wait until all the slides have been processed. Consequently, this means that automating the processing of Pap staining may not actually produce any time efficiencies.

It is an object of the invention to provide an apparatus which reduces or substantially obviates the above mentioned problems.

According to a first aspect of the invention, there is provided a method of preparing cytological specimens for analysis, which comprises the steps of: providing cytological material in a specimen container, adding anti-coagulant preservative to the specimen container, and receiving the cytological material and anti-coagulant in an automated apparatus for pre-analysis preparation. The anti-coagulant preservative is selected to inhibit cellular activity and maintain cellular morphology without killing cells.

The benefit of this method is that the cytological cells are preserved in an active state, with little or no degeneration prior to staining. Use of an anti-coagulant preservative in the preparation of cytological specimens leads to minimal damage or distortion to the structure, and architectural and/or morphological appearances of the cytological cells, as tends to occur using an alcohol based preservative. Cells subjected to an alcohol based preservative become fixed and effectively die, making the cells difficult to manipulate for successful performance in a wide range of diagnostic techniques.

Cells harvested from the body and preserved using the anti-coagulant and processed in this manner perform better in any subsequent treatment they are put through. Histochemical tests, PAS, GMS, ZN and Perl's Prussian Blue and ancillary tests such as HPV testing, Immunocytochemical tests, flow-cytometry and the like can be performed on harvested cells with an excellent staining outcome.

Preferable and/or optional features of the first aspect of the invention are set forth in dependent claims 2 to 39.

According to a second aspect of the invention, there is provided an apparatus for automatically preparing pre-analysis cytological specimens comprising: receiving means for receiving at least one deposit of a cytological specimen, first and second staining stations, and transfer means for transferring a portion of the or each deposit from the receiving means to the first staining station and/or a portion of the or each deposit to the second staining station, the first staining station being operable for a Papanicolaou staining technique and the second staining station being operable for a Papanicolaou staining technique and at least one further staining technique.

The apparatus is advantageous because it is able to receive both gynaecological samples and non-gynaecological samples for automated staining. This represents a significant improvement over existing equipment which can only automate the processing of Papanicolaou staining techniques. A portion of each specimen can be sent for Papanicolaou staining, non-Papanicolaou staining, or both Papanicolaou and non-Papanicolaou staining for simultaneous processing. This enables the medical professional to quickly obtain a comprehensive overview of the health of the patient without the delay and financial burden of having to send samples to different laboratories for preparation.

For effective practice and confidence of quality assurance, it is beneficial that a variety of staining techniques are carried out on non-gynaecological samples. The apparatus is configured to allow a wide range of staining techniques to take place in the same location. Multiple staining protocols can be carried out on the same machine within the same batch of samples.

Preferable and/or optional features of the second aspect of the invention are set forth in dependent claims 41 to 78.

According to a third aspect of the invention, there is provided a method of preserving cellular material comprising the steps of a) placing the cellular material in a container, b) adding to the container a solution comprising 0.9 g sodium chloride, 99 ml distilled water and 1 ml concentrated formaldehyde, or any other quantities substantially in those proportions.

According to a fourth aspect of the invention, there is provided a method of preserving cellular material comprising the steps of a) placing the cellular material in a container, b) adding to the container a solution comprising 50 ml heparin sodium, 50 ml saline, 5 g chloral hydrate and 0.1 g citric acid, or any other quantities substantially in those proportions, and c) freezing the cellular material suspended in the solution.

For a better understanding of the present invention, and to show more clearly how it may be carried into effect, reference will now be made, by way of example only, to the accompanying drawings, in which:

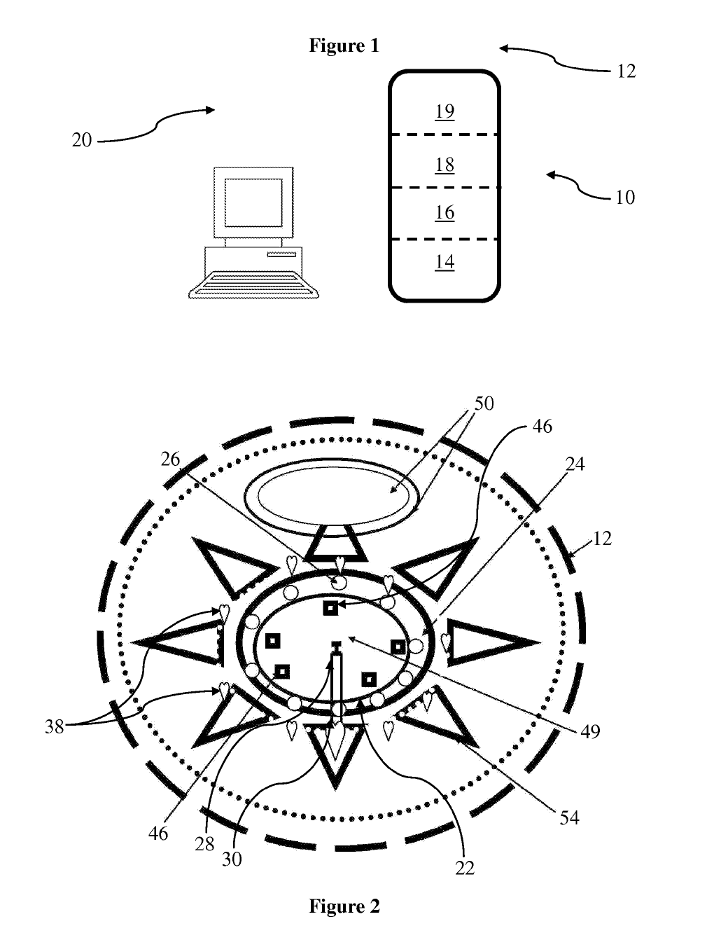

FIG. 1 shows a schematic general overview of the apparatus according to the second aspect of the invention, showing in particular the processor that controls the various processes within the apparatus;

FIG. 2 shows a schematic plan view of a base level of the apparatus of FIG. 1;

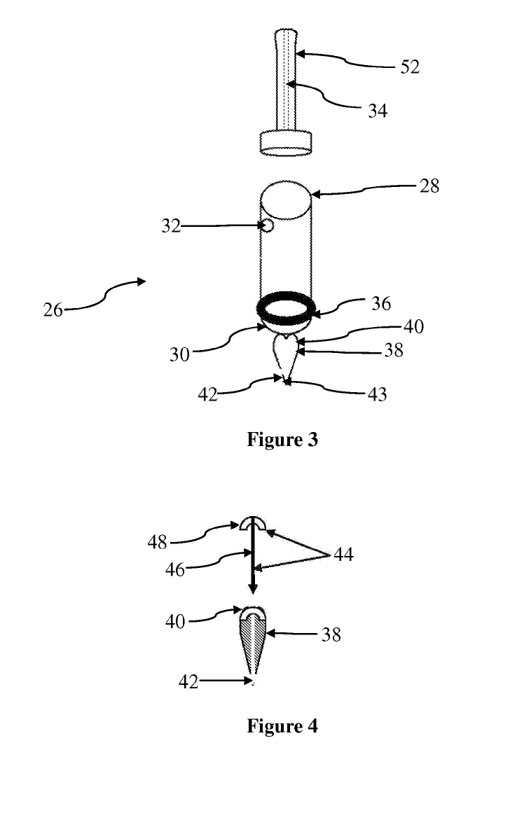

FIG. 3 shows a schematic front view of first and second receptacles of the apparatus;

FIG. 4 shows a schematic close-up of the second receptacle receiving agitation means;

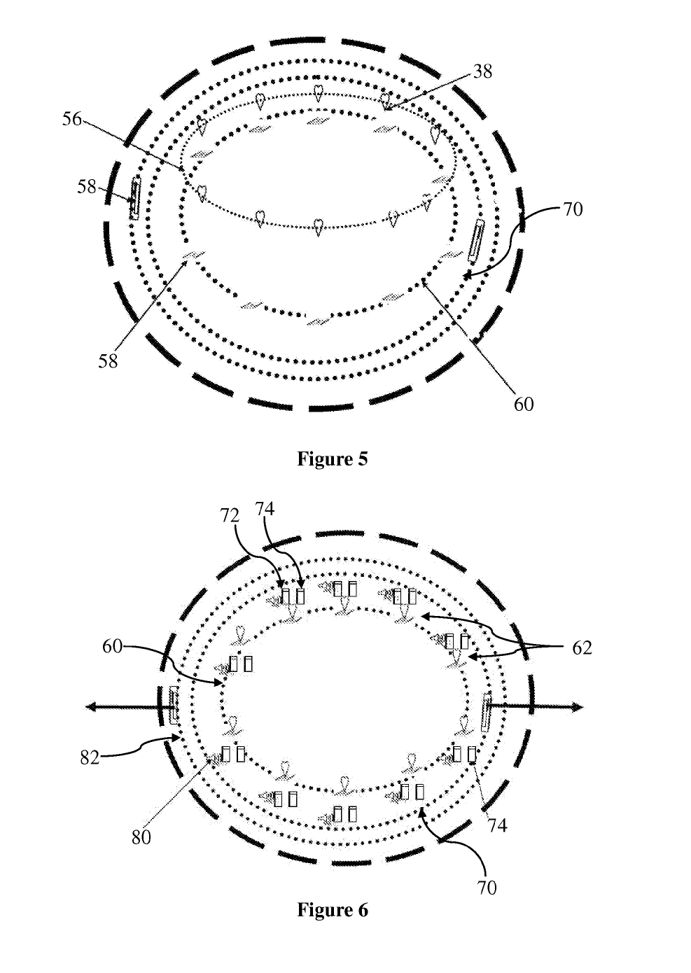

FIG. 5 shows a schematic plan view of a first level of the apparatus of FIG. 1;

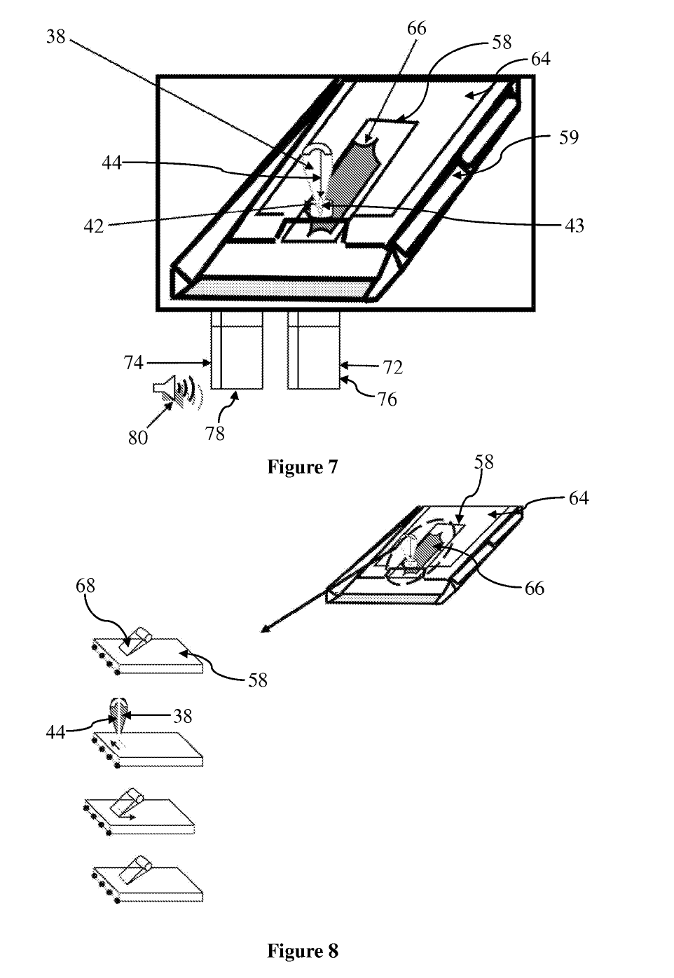

FIG. 6 shows the schematic plan view of the first level with samples being transferred between a fixing stage and a staining stage;

FIG. 7 shows a schematic close-up of a smearing unit of the apparatus in the second aspect of the invention, and shows in particular first and second fixing stations;

FIG. 8 shows a schematic close-up of the smearing unit in operation;

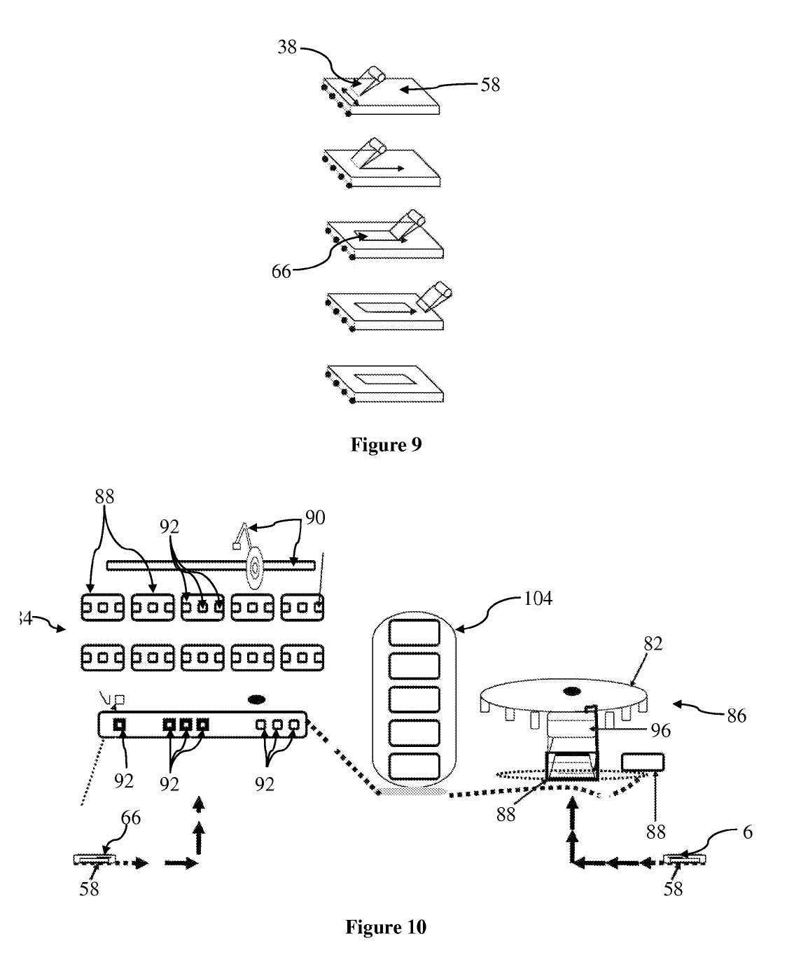

FIG. 9 shows a schematic close-up of a spreading member of the smearing unit in action;

FIG. 10 shows a schematic overview of a staining station in the second aspect of the invention in action;

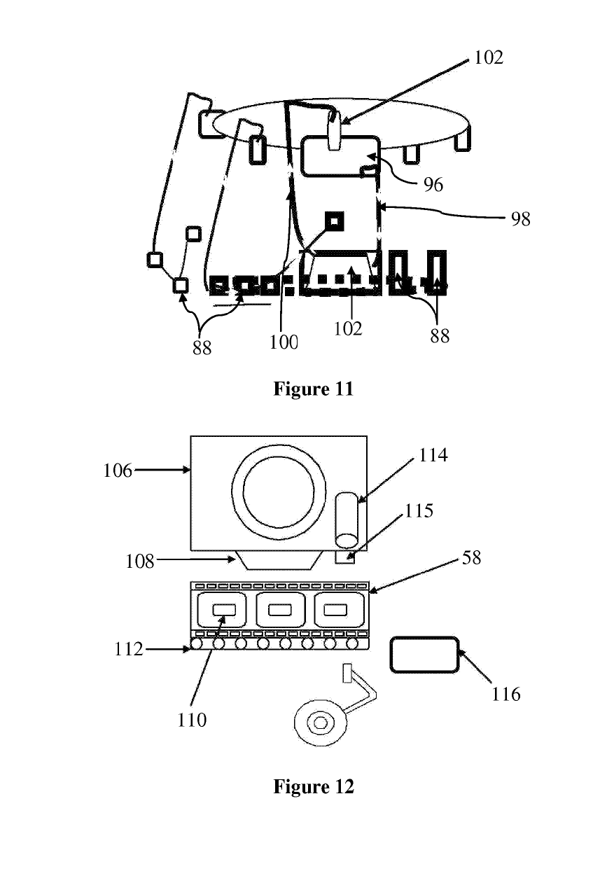

FIG. 11 shows a schematic view of staining fluid stored in a staining tank and in fluid connection with a staining trough;

FIG. 12 shows a schematic view of a coverslipping unit of the apparatus in the second aspect of the invention; and

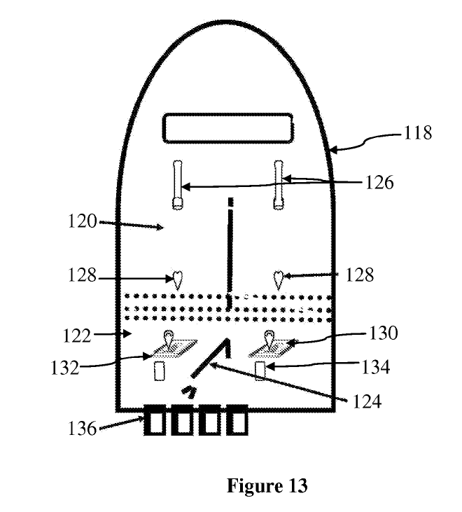

FIG. 13 shows a schematic overview of a satellite sub-apparatus of the apparatus in the second aspect of the invention.

Referring firstly to FIG. 1, an apparatus for automatically preparing pre-analysis cytological specimens is indicated generally at 10. The apparatus comprises a housing 12 which houses a plurality of preparation stations. The preparation stations are arranged over the following four levels: a base level 14 in which pre-staining preparation such as purification takes place; a first level 16 in which smearing and fixing takes place, a second level 18 in which staining takes place, and a third level 19 in which mounting/coverslipping takes place. Operation of the apparatus is controlled by a processor, for example a personal computer 20.

The processor 20 controls the operation of the apparatus 10. It includes labelling means for labelling samples with a computer-readable identifier (for example, a barcode), and a scanner for estimating the number of epithelia cells present in a sample.

Referring now to FIG. 2, the base level comprises a tubular carousel 22 having a plurality of reception ports or sockets 24 extending around the carousel 22. Each reception port 24 is configured for receiving and supporting a receptacle or barrel 26 containing a deposit of a cytological specimen. Preferably, the number of reception ports 24 provided is in the range of 5 to 25. More preferably, there are between 10 and 20 reception ports 24 disposed around a peripheral extent of the carousel 22.

As shown in FIG. 3, each receptacle 26 is elongate and tubular, and has first and second ends 28, 30. Preferably, each receptacle 26 has a circular lateral cross-section so as to be receivable in one of the reception ports 24 in a close fitting relationship. Each receptacle 26 may be or include glass to facilitate a macroscopic visual inspection of the receptacle 26 contents. The purpose of the receptacle 26 is to receive cytological material transferred from a specimen container (not shown). The specimen container is the container used to temporarily store a cytological sample at the point of patient contact, for example extracted by a physician, and typically takes the form of a glass vial.

Three volumetric sizes of receptacle 26 may be provided: large, medium and small, the large size having a volumetric capacity of, for example, approximately 50 ml, is particularly suitable for containing samples which have a relatively high viscosity, for example sputum bronchial wash. Samples having a high density of fibroids can also easily be accommodated within the large receptacle. The medium sized receptacle is useful for specimens having a moderate consistency or viscosity. The small sized receptacle, having a volumetric capacity of, for example, approximately 10 ml, is especially useful for clear fluids containing a small number of epithelial cells, e.g. cerebrospinal fluids.

A fluid inlet 32 for receiving diluent is provided at or adjacent to the first end 28 of each receptacle 26. Proximate the second end 30 of at least one of the receptacles 26 is a filter band 36. The filter band 36 may be adapted to filter out red blood cells from the specimen, for example, by having relatively large apertures. It is preferable that the filter band 36 is made from a robust material so as to be capable of withstanding the stress of repeated filtration without significant damage.

A further receptacle or pouch 38 is detachably attached to the second end 30 of each first said receptacle. The further or second receptacle 38 on a large first receptacle may have a volumetric capacity of 0.71 ml. The second receptacle on a small first receptacle may have a volumetric capacity of 0.4 ml. The second receptacle is at least partially flexible to assist with the ejection of epithelial cells therefrom.

Each second receptacle 38 is preferably elongate and tubular and has first and second ends 40, 42. The first end 40 of the second receptacle 38 is releasably engageable with the second end 30 of the first receptacle 26. The second receptacle 38 may be engageable with the first receptacle 26 with a screw thread arrangement. Detaching means (not shown) are provided for detaching a second receptacle 38 from a corresponding first receptacle 26. Typically, the detaching means includes one or more cutting members. The second end 42 of the second receptacle 38 houses a valve 43, which is preferably digitally controllable. The valve 43 is configured to selectively permit the passage of epithelial cells therethrough.

A microbore may be provided at or within the first end 40 of the second receptacle 38 through which epithelial cells are received from the first receptacle 26 when the first and second receptacles 26, 38 are coupled together. The microbore prevents the ingress of non-epithelial material into the second receptacle 38. As such, the aperture of the microbore is measurable at a cellular level.

Agitation means 44 for agitating epithelial cells in each second receptacle may also be provided. Preferably, the agitation means takes the form of a Pasteur's pipette stem 46 with a closure member 48 attached to one end thereof. The Pasteur's pipette stem 46 is at least partially resiliently compressible.

As shown in FIG. 4, the Pasteur's pipette stem 46 is receivable in the first end 40 of a second receptacle 38 that has been detached from a corresponding first receptacle 26 using the detaching means. The closure member 48 covers and preferably seals the first end 40 of the second receptacle 38, thereby enclosing epithelial cells received from the first receptacle 26 within. This arrangement is beneficial as it prevents leakage during subsequent transportation; it also prevents the ingress of airborne contamination into the second receptacle. Furthermore, the closure member 48 provides an easily accessible surface with which the Pasteur's pipette stem 46 can be manipulated. The Pasteur's pipette stem 46 extends through the second receptacle 38 and terminates proximate the valve 43.

The carousel 22 is moveably mounted within the housing 12 and is preferably axially removable from the housing 12 so that further first and second receptacles 26, 38 can be added to the carousel 22, as and when required. However, further first and second receptacles 26, 38 may be added to the carousel 22 whilst the carousel is in situ in the apparatus 10. The carousel 22 is preferably moveable in at least the following two modes: a first mode, in which the carousel 22 is rotated about a central axis of rotation, and a second mode, in which the carousel 22 is able to gently reciprocate back and forward about the same axis of rotation. The two movement modes facilitate centrifugation and swinging of receptacles attached to the carousel in the reception ports. Centrifugation and swinging are important processes to occur during the preparation of pre-staining cells. Both processes facilitate mixing or blending of the cytological specimen with other fluids, and are described in further detail below.

Each reception port 24 is provided as an elongate channel which is semi-circular in lateral cross-section. Alternative lateral cross-sections may be used provided that the receptacle has a corresponding lateral cross-section. Each reception port 24 comprises holding means for holding at least a portion of a first receptacle 26 and/or a second receptacle 38.

The holding means are preferably provided at opposing ends of each reception port 24. The holding means may comprise a pair of arms, into which a first receptacle 26 is receivable. The holding means are preferably at least in part arcuate. The holding means may alternatively include a single clamp member. When the carousel 22 operates in the swinging mode, the holding means only supports the first receptacle 26 at one end of the first receptacle 26. During centrifugation, the holding means firmly supports both ends of the first receptacle 26.

Each reception port 24 further comprises at least one sensor for detecting the quantity and/or absence of red blood cells, mucous, neutrophils and/or epithelial cells in a specimen initially hosted by a first receptacle 26.

As best seen in FIG. 2, a plurality of storage compartments or tanks 46 for storing a non-epithelial material removing agent, such as a mucous withdrawing agent, a red blood cell removing agent or a neutrophils removing agent, are disposed radially inwardly of the carousel 22. One of the plurality of storage compartments 46 may be used to store diluents. The storage compartments 46 are provided within a static central core 49 of the apparatus. When the carousel 22 is stationary, each storage compartment 46 is in fluid communication with one or more of the reception ports 24.

Each reception port 24 further comprises first and second connection means (not shown). The first and second connection means bridge the gap between each first receptacle 26 and each reception port 24. Non-epithelial material removing agents and diluents are deliverable to the first receptacle 26 via the fluid inlet 32 using the first connection means. Supernatant is drainable from the first receptacle via the fluid outlet 34 using the second connection means. The second connection means is in fluid communication with a drain. The first and second connection means are preferably rigid sections of fluid conduit.

A flushing element 50 is disposed proximate the carousel 22. The flushing member includes a piston 52 which is suitably dimensioned to be receivable within the first end 28 of a first receptacle 26. The piston 52 is axially moveable relative to the carousel 52. The role of the flushing element 50 is to flush out non-epithelial matter from the specimen through the filter 36. A fluid outlet 34 is provided through the longitudinal central axis of the piston.

A plurality of cap storage units 54 are also disposed radially outwardly of the carousel, proximate the storage compartments 46. Each cap storage unit 54 accommodates a stack of caps used to cover the first end 28 of the first receptacle 26, preferably using a threaded screw arrangement. Each cap storage unit 54 is adapted to dispense a cap into one or more reception ports 24 and onto a first receptacle 26 therein.

As best seen in FIG. 5, a first annular conveyor 56 for transferring second receptacles 38 to the first level 16 of the apparatus 10 is provided. To facilitate transportation, the first annular conveyor 56 comprises a plurality of sockets configured for receiving, holding and releasing second receptacles. The first annular conveyor 56 is moveable axially between the base level 14 of the apparatus and the first level 16 of the apparatus 10.

A slide cartridge (not shown) for temporarily storing a plurality of specimen slides 58 is provided in the first level 16 of the apparatus 10. Preferably, the slide cartridge houses approximately 10 slides. Each slide 58 may be a ‘microslide’ with exemplary dimensions of 3×4 cm. The slide cartridge gently dispenses slides 58 one by one onto a second annular conveyor 60, described in further detail below. The slide cartridge may be replenishable in situ or it may be removable from the apparatus 10 to facilitate replenishment of specimen slides 58.

The slide cartridge preferably hosts an identification means so that the identity of a sample ejected onto a slide 58 is not lost at this stage. The identification means tags each slide 58 dispensed from the slide cartridge, for example using a barcode applied to the slide 58 with a self-adhesive label. It is important that the self-adhesive should be chemical and heat resistant in order to withstand the rigours of the fixing process.

Referring to FIG. 6, at least one smearing unit 62 for spreading epithelial cells on a specimen slide 58 is also disposed in the first level 16 of the apparatus 10. Each smearing unit 62 is arranged to receive a second receptacle 38 containing epithelial cells which has been transferred from the base level 14 to the first level 16 using the first annular conveyor 56. Each smearing unit 62 is also arranged to receive specimen slides 58 from the slide cartridge on the second annular conveyor 60.

Referring now to FIGS. 7, 8 and 9, each smearing unit 62 comprises a platform 64 onto which a specimen slide 58 is delivered by the second annular conveyor 60. The smearing unit 62 further comprises actuation or ejection means, such as a clamp, for applying external pressure to each second receptacle 38 to eject epithelial cells 66 therefrom. Co-ordinated operation of the valve 43 in the second receptacle 38 and the ejection means results in a portion of the epithelial cells 66 from within the second receptacle 38 being deposited on an adjacent specimen slide 58.

Each smearing unit 62 also includes a spreading member 68, such as a blade, for gently spreading the portion of cells 66 over the specimen slide 58. The spreading member 68 operates at a gentle steady speed to allow the smeared cells 66 to cover a pre-set extent of the specimen slide 58. The spreading member 68 and the platform are separated by a gap of at least 5 microns. This gap helps to control the thickness of the layer of cells 66 deposited on the specimen slide 58. The spreading member 68 may be moveable relative to the platform 58, or the platform 58 may be moveable relative to the spreading member 68. A compressible rubber edge strip 59 is provided around the smearing unit 62. This can be compressed as the platform 58 is moved relative to the spreading member, in order to precisely control the gap between the spreading member 68 and the platform 58 and hence the thickness of the layer of cells 66.

The smearing unit 62 also includes a labeling unit to automatically label each slide with a chemical resistant ink before smearing.

The second annular conveyor 60 is provided to transfer at least one specimen slide 58 from the slide cartridge to the or each smearing unit 62. To facilitate transportation, the second annular conveyor 60 comprises a further plurality of sockets configured for receiving, holding and releasing specimen slides 58. The second annular conveyor 60 is moveable radially within the first level 14 of the apparatus 10.

A third annular conveyor 70 for transferring smeared samples from the or each smearing unit 62 to first or second fixing units 72, 74 is also provided. To facilitate transportation, the third annular conveyor 70 comprises a yet further plurality of sockets configured for receiving, holding and releasing smeared specimen slides 58. The third annular conveyor 70 is moveable both axially and radially within the first level 16 of the apparatus 10.

As best seen in FIG. 7, the first fixing unit 72 is arranged for the chemical fixing of smeared epithelial cells 66 on a specimen slide 58. The first fixing unit 72 comprises a fluid bath or trough 76 for receiving an alcohol base fixative. The fixative may be 95% to absolute alcohol. A second fixing unit 74 is arranged for air-drying smeared epithelial cells 66 on a specimen slide 58. The second fixing unit 74 includes a recess or trough 78 and an air-dryer 80 located adjacent to the recess 78. Both troughs are dimensioned to receive a specimen slide 58.

As best seen in FIG. 6, a fourth annular conveyor 82 for transferring specimen slides 58 supporting fixed smeared epithelial cells 66 from the first level 16 to the second level 18 of the apparatus 10 is provided. To facilitate transportation, the fourth annular conveyor 82 comprises a yet further plurality of sockets configured for receiving, holding and releasing specimen slides 58. The fourth annular conveyor 82 is axially moveable between the first level 16 of the apparatus 10 and the second level 16 of the apparatus 10. The fourth annular conveyor 82 is also preferably radially moveable.

First and second staining stations 84, 86 are provided in the second level 16 of the apparatus 10. Slides 58 exiting from the first fixing unit 72 are transferred to the first staining station 84, whereas slides 58 exiting from the second fixing unit 74 are transferred to the second staining station 86. The first staining station 84 is operable for a Papanicolaou staining technique and the second staining station 86 is operable for a Papanicolaou staining technique and at least one further staining technique. The first staining station 84 is preferably dedicated to receiving gynaecological samples and the second staining station 86 is dedicated to receiving non-gynaecological samples.

The further staining techniques may include any one or more of the following staining techniques: May Grunwald Giemsa (MGG), Periodic Acid Schiff's (PAL), Perls' Prussian Blue (Perl's), Gomoris Methanamine Silver (GMS), Groscott's methods for Silver, Alcian Blue, Ziehl-Neelson (ZN for Acid Fast), and Oil Red 0. The various staining protocols or programs are pre-programmed onto the processor 20.

Turning now to FIG. 10, the first staining station 84 comprises a plurality of staining troughs 88 and a handling member 90 for transferring a specimen slide 58 from trough 88 to trough 88. The troughs 88 are preferably aligned end to end in series. Each trough 88 has a pressure controlled water inlet and outlet for rinsing smears after each stage of the staining protocol if necessary. Each trough 88 includes at least one bath 92 for containing a particular staining fluid or reagents. The or each bath 92 in each trough 88 will typically contain the same staining fluid or reagent.

The second staining station 86 is arranged in a similar way. However, the staining tanks 96 in the second staining station 86 primarily contain staining fluid and reagents particular to non-Papanicolaou staining techniques.

Each staining trough 88 and/or bath 92 may include a transparent window for facilitating a visual inspection of the sample during staining. To this end, the apparatus may include one or more USB connection ports for receiving a USB digital microscope.

At least one of the troughs 88 may be temperature controlled, particularly on the second staining station 86.

A plurality of staining tanks 96 for storing staining fluid or fluidic reagents is provided in the second level 16. The fluids can be, for example, water, alcohol, xylene, haematoxylin, OG6 and EA50, depending on which staining techniques are required.

As indicated in FIG. 11, each staining tank 96 is fluidly connected with at least one of the staining troughs 88 via flow and return connecting means, 98, 100. Preferably, a disposable filter 102 is provided in each return connecting means 100, upstream of the respective staining tank 96. In such an arrangement, staining fluid can be delivered to a staining trough 88 for a single application or for prolonged use. The staining fluid can then be removed from the staining trough 88 and pumped back to the appropriate staining tank 96 through the filter 102. As such, the staining fluids can be recycled. The staining troughs 88 can discard their contents once or twice a day (or week if during periods of low activity) as required, and simply be refilled from the staining tanks 96. A sensor in each staining tank 96 closely monitors the staining fluids and reagents to assess their suitability for further staining processes after each round of staining. If no longer suitable, the particular fluid is discarded and replaced.

Preferably, each staining station 84, 86 further comprises a cleaning device for facilitating thorough rinsing and cleaning of the staining station 84, 86 in between successive use of different staining fluids. This prevents carryover into a follow-on staining fluid and contamination of the remaining stock of staining fluids. The cleaning device can be programmed to carry out daily disinfection and/or weekly disinfection.

Each staining tank 96 may include an access port accessible through the housing 12 of the apparatus 10 for facilitating replenishment of staining fluid therewithin.

Although the staining tanks 96 have been described as being internal of the apparatus 10 housing, they may alternatively be located externally of the housing 12 and connected to the staining troughs 88 using conduit.

The apparatus 10 may further comprise at least one accumulator 104 or ‘hangar’ to temporarily accommodate fixed smeared samples before being taken to the staining stations 84, 86. For example, if the current staining protocol is taking longer than the purification and smearing processes and a backlog of samples is beginning build up, then fixed smeared samples may be stored in the accumulator 104 until such a time as one or other of the staining stations 84, 86 is free.

As shown in FIG. 12, a coverslipping unit 106 is located on the third level 19 of the apparatus 10. The coverslipper unit 106 comprises a storage compartment 108 for temporarily housing a plurality of coverslips 110, a linear conveyor 112 for transporting stained slides and a mounting unit 114 for applying a coverslip 110 dispensed from the storage compartment 108 through a nozzle 115 onto a stained slide as it passes on the linear conveyor 112. The coverslipping unit 106 may be configured to receive slides from one or other or both of the first and second staining units 84, 86. The coverslipping unit 106 operates in a conventional manner and includes known coverslipping components.

A further accumulator 116 may be provided in the second level 18 to accommodate a plurality of stained specimens before they are transferred to the coverslipping unit 106.

Referring now to FIG. 13, the apparatus 10 may further comprise a satellite sub-apparatus 118 specifically intended for use with Fine Needle Aspirant (FNA) biopsies in FNA Clinics and Wards. The satellite apparatus 118 has two compartments: upper and lower compartments 120, 122. The upper compartment 120 contains a free microfuge with two positions for holding and clamping purifiers. The lower compartment 122 houses a smearing unit substantially as described previously with respect to smearing unit 62, a slide transfer handling mechanism 124, and a plurality of staining troughs substantially as described previously with respect to troughs 88.

The satellite apparatus 118 works in a similar way as the main apparatus 10. Initially, the collected sample may be purified or it may be poured directly into a first receptacle and then mounted in a carousel. The carousel is then balanced. The satellite apparatus 118 is activated to then compress the sample using a flushing member 126 to approximately 1 ml. Supernatant fluid is extracted through a filter as in the parent machine. The first receptacle is then automatically refilled with diluents, mixed and spun. Epithelial cells are transferred directly into a second receptacle 128. The second receptacle 128 is then detached from the first receptacle, sealed by a closure member and shaken gently to mix emollient with the specimen deposit before being plugged into a smearing unit 130 at the lower level 122. The deposit is then discharged on a slide 132 and the smear is made as in the parent machine. The smear is then air-dried in a trough 134. The smear is subsequently stained in staining troughs 136 using Diff Quick technique for MGG. A coverslip is then applied onto the slide.

The advantage of the satellite sub-station 118 is that it provides surgeons with the facility to take a biopsy and make an almost immediate diagnosis of a patient's condition. Theoretically, in theatre, a frozen section test is supposed to be completed in approximately 10 minutes because it is performed on a tissue taken out of a patient by a surgeon during a surgical operation. It is meant to inform the surgeon whether or not a suspicious looking tissue or organ is undergoing a pathologic process or not. The tissue is taken in theatre and sent to a laboratory immediately for frozen section. The time-lag between sample transportation to the laboratory, preparation of sections, compiling a report and returning the report to the operating theatre make the 10 minute target unrealistic.

The satellite sub-station 118 provides the facility to make a diagnosis in around 10 min, with the breakdown as follow: 1) Loading the sample—1 min, 2) Compression—2 min, 3) Spinning—3 min, 4) Smear making—1 min, 5) Staining—2 min, and, 6) Operational pauses—2 min.

To facilitate use of the satellite sub-station 118 and preparation of a biopsy, a specially adapted syringe may be provided. Traditionally, FNA samples are taken with a conventional syringe and it is essential to expel any excess fluid taken quickly to avoid clotting of the sample. Clots frequently occur within a short period of time, and the smaller the sample, the higher the risk of clotting. Sometimes, the aspirated samples clot immediately and get stuck in the barrel, rendering the sample useless.

The specially adapted syringe is internally coated with heparin, which would prevent clotting in any form. No matter how small the sample, cells in the syringe can be still be successfully harvested and as much as possible be presented in their morphological state.

In use, cytological material is provided in a specimen container at point of patient contact. Anti-coagulant is added to the cytological material. The anti-coagulant acts as a preservative to the cytological material whilst the sample is being transported to an apparatus for pre-cytodiagnosis preparation. Anti-coagulants which may be used include: 1) heparin, which is used to preserve urine, peritoneal ascetic fluid and ovarian fluid, 2) EDTA, which is used to preserve mucous and fibrinoid samples e.g. sputum, bronchial larvage, washings, and any other sample that has a heavy texture or viscosity, and 3) a combination of heparin sodium, chloral hydrate and citric acid. Heparin sodium choral hydrate is a cytoplasmic preservative and citric acid is a nuclear sharpener. It is found that a mixture comprising 50 ml heparin sodium, 50 ml saline, 5 g choral hydrate and 0.1 g citric acid forms a preservative suitable for preserving cells in urine and serous fluids whilst preventing clotting of the extracellular medium surrounding the cells. EDTA prevents coagulation as well as degrading fibrin and mucous without destroying the cells of interest.

The specimen container may be pre-charged with the anti-coagulant or the anti-coagulant may be added to the container after the cytological material is inserted therein.

The specimen container is then tagged using identification means so that the identity of the specimen container is unique and traceable. The identification means typically labels the specimen container with a barcode. The identification means assists with the identification and traceability of a sample from the initial specimen container to the final specimen slide ready for cytodiagnosis.

The medical professional optionally categorizes the cytological material according to the viscosity and/or level of blood staining, depending on the nature of the sample. This information relating to the sample is entered into a log, for example, on the personal computer. Additional information such as patient identification details, name of the physician taking the sample, the nature of the sample (e.g. whether it is gynaecological or non-gynaecological) and the like, is also entered onto the computer 20.

The sample may be exposed to a scanner which is connected to the computer 20. The scanner provides a quantitative estimation of the number of epithelia cells present in the sample. This estimation is used by the computer 20 to make suitable adjustments to the purification stage of the apparatus 10 depending on the estimated level of cellularity of the sample.

The sample is then manually transferred into a preparation receptacle (or a first receptacle 26) which is specially adapted for the preparation process, i.e. it has a second receptacle 38 detachably attached at one end thereof. The volumetric capacity of the receptacle 26 may be selected according to the viscosity and/or level of blood staining in the sample. The first and second receptacles 26, 38 are then tagged using the identification means, for example, a duplicate barcode, applied using a self-adhesive label. The first and second receptacles 26, 38 are then inserted into the carousel 22 and the carousel 22 mounted on the apparatus 10.

Each reception port 24 of the carousel 22 does not have to be full with specimen receptacles 26, 38 before the apparatus 10 can operate since the apparatus 10 is arranged to carry out an auto-balancing procedure on the carousel 22, as and when required or at regular intervals. The aim of the balancing procedure is to achieve an even mass distribution across a diametric extent of the carousel 22.

The sample then undergoes a purification or enrichment process to remove non-epithelial materials from the sample. The purification process includes several stages, including cellular washing, flushing, swinging and centrifugation. One or more stages of the purification process may be repeated depending on the composition of the sample.

One of the at least one sensors in each reception port 24 detects the level of mucous, red blood cells, neutrophils and epithelial cells in the sample. One or more of the following agents are then added to the sample accordingly: mucous withdrawing agent, netutrophils withdrawing agent and red blood cell withdrawing agent. Each individual agent couples with its corresponding non-epithelial material to remove the unwanted calls from the sample, i.e. to ‘wash’ the sample. The quantity of mucous, red blood cells, neutrophils and epithelial cells in the sample is checked at regular intervals and further agents added if required.

Diluent may also be added at any time to reduce the viscosity of the sample as required.

A cap is attached to the first end 28 of each first receptacle 26 prior to any swinging and centrifugation to prevent leakage of cells from the first receptacle 26.

The sample undergoes swinging so as to mix or blend the sample with any diluents that have been added.

During centrifugation, blood, mucus and other debris from the sample are separated from the diagnostic material, to create an enriched cellular sample.

Flushing occurs when the flushing member enters the first receptacle to flush non-epithelial material through the fluid outlet.

The sensor establishes when all the red blood cells, mucous and neutrophils, as well as any other non-epithelial material have been removed from the sample.

The sensor establishes whether there is a sufficient quantity of epithelial cells in the sample for further processing. If an insufficient quantity of epithelial cells is detected, the apparatus operator is alerted to the particular first and second receptacles using alert means, such as an audible alarm. The alert means may be a combined visual and audible alert, or simply a visual alert.

The user may be prompted to establish whether there is further sample left in the original specimen container to add to a further first receptacle, before beginning a further purification cycle on the new sample. Alternatively, the processor 20 may be programmed to halt the apparatus 10 for a period of say 3 minutes before continuing with the initial sample. As a further alternative, the processor 20 may be programmed to automatically eject the initial sample from the carousel 22 so as not to delay processing of other samples within the apparatus 10.

A final spin sends the epithelial cells to the bottom of the first receptacle 26.

Finally, supernatant is drained from the first receptacle 26, leaving only epithelial cells remaining. The epithelial cells are then transferred into the adjacent second receptacle 38.

After purification is complete, epithelial and lymphocyte cellularity is assessed by a flow cytometry scanner. Cellularity measurements are saved for use during the smearing process. At this stage, a more accurate assessment of cellularity can be carried out than the estimate obtained by the scanner before purification.

The second receptacle 38 is detached from the first receptacle 26 using the detaching means. The Pasteur's Pipette stem 46 is inserted into the first end 40 of the second receptacle 38, which is closed by the closure member 48. The Pasteur's pipette stem 46 is manipulated to apply a gentle rhythmical agitation to the epithelial cells to ensure that an even (and unclumped) cell distribution before smearing takes place. The first annular conveyor 56 then transfers each second receptacle 38 from the base level 14 to the first level 16 into one of the at least one smearing units 62. The second receptacle 38 is then transferred into the smearing unit 62 and the first annular conveyor 56 is returned to the base level 14.

A measured portion of the epithelial cells 66 from the second receptacle 38 is then injected onto a specimen slide 58, which has been carried to the smearing unit 62 from the slide cartridge on the second annular conveyor 60. The spreading member 68 spreads out the deposit of epithelial cells 66 over the specimen slide 58 in a mono-cellular layer. The dimensions of the cell smear 66 are determined taking into account the cellularity measurements obtained after purification. If the cellularity of the sample is high, the smear may be of a greater length than for a low cellularity sample.

The specimen slide 58 is then transferred to one of the fixing units 72, 74 using the third annular conveyor 70. Depending on the quantity of epithelial cells remaining in the second receptacle, additional deposits of the same cytological material may be subsequently injected onto additional specimen slides.

If the epithelial cells 66 are gynaecological, then the specimen slide 58 is sent to the first fixing unit 72 for chemical fixing. If the epithelial cells 66 are non-gynaecological, then the specimen slide 58 is sent to the second fixing unit 74 for air-drying.

The specimen slide 58 is then transported to the first or second staining station 84, 86 accordingly, using the fourth annular conveyor 82. The desired staining protocol then takes place.

Finally, the stained slide is transferred to the coverslipping unit 106.

To produce a batch of 20 smears takes approximately 90 min with the following breakdown:

### Purification

Swinging—2 min, Spinning—5 min, Flushing—2 min. Repeat process.

Sub-total—18 min

### Smearing

Mixing of deposit to achieve smooth consistency—1 min, Dropping deposit on one slide and spreading—2 min, Dropping deposit on further slide and spreading—2 min, Operational Pauses—2 min

Sub-total—7 min

This indicates that to prepare 40 slides only (without staining) would take 25 min.

### Fixing and Staining

Fixation—20 min, routine staining—40 min, coverslipping—5 min

Sub-total—65 min

The combined total of purification, smearing, fixing and staining is approximately 90 min.

This indicates that to complete the whole process, i.e. preparation of smears, fixing, staining and coverslipping would take approximately 90 min. The apparatus could therefore complete 5 batches over an 8 hr working period. During extended working hours, between for example 8 am and 8 pm, with good laboratory experience and effective planning, the apparatus can feasibly process a workload of 200 cervical smears and 50 non-cervical smears per day.

Where the quantity of cells collected is greater than the quantity needed to produce the required smears, the excess cells may be stored for future use.

Excess cells may be stored in 1% formal saline, comprising 0.9 g sodium chloride, 99 ml distilled water and 1 ml concentrated formaldehyde. A small amount of this solution is dispensed on top of the deposit to form a suspension, and the suspension may be kept for at least two years in a plastic container.

Alternatively, excess cells may be freeze-dried in citreous heparin, comprising 50 ml heparin sodium, 50 ml saline, 5 g chloral hydrate and 0.1 g citric acid. After being mixed with citreous heparin, the deposit is frozen, and is preserved for around one year.

In summary, the apparatus is designed to process all bodily fluids, including cervical smears, for cytological investigations. Cells processed using the apparatus are less exposed to procedural stress to minimise alterations to their structures and architectures. Extra- and intra-cytoplasmic components, that are not only vital to cellular investigations but sometimes holds clues to diagnostic dilemmas in difficult situations, are also well preserved and presented for investigations.

The apparatus is a stand-alone machine that harvests cells from various media consistencies by effectively removing the media they come from, washes the cells, prepares monolayer smears of not more than 5 micron from them, fixes or air-dried the smears, stains them with routine Papanicolaou and Romanowsky techniques.

The apparatus can retrieve cells from any bodily fluid and produce excellent artefact free thin layer smears therefrom.

Automation of the pre-cytodiagnosis preparation process provides high quality and standardised results, enabling easier interpretation and reproducibility.

1.-81. (canceled)

82. A method of preparing cytological specimens for analysis, which comprises the steps of:
a. Providing cytological material in a specimen container,
b. Adding anti-coagulant preservative to the specimen container,
c. Receiving the cytological material and anti-coagulant in an automated apparatus for pre-analysis preparation,
the anti-coagulant preservative being selected to inhibit cellular activity and maintain cellular morphology without killing cells.

83. A method as claimed in claim 82, in which the anti-coagulant preservative comprises at least in part of heparin sodium chloral hydrate, and at least in part of citric acid.

84. A method as claimed in claim 83, in which the anti-coagulant preservative comprises substantially 50 ml heparin sodium, 50 ml saline, 5 g chloral hydrate, and 0.1 g citric acid or any other quantities substantially in that proportion.

85. A method as claimed in claim 82, in which the anti-coagulant is added to the specimen container before the cytological material is provided therein.

86. A method as claimed in claim 82, further comprising the step of categorizing the cytological material according to viscosity and/or level of blood staining.

87. A method as claimed in claim 82, further comprising the step of transferring the cytological material and anti-coagulant from the specimen container to a preparation receptacle, the receptacle having a fluid inlet for receiving diluents and a fluid outlet for draining supernatant.

88. A method as claimed in claim 87, in which the receptacle has a flexible second said receptacle detachably attached at one end of the first said receptacle.

89. A method as claimed in claim 88, further comprising the step of purifying the cytological material in a purification procedure to remove unwanted non-epithelial materials.

90. A method as claimed in claim 89, in which said purification procedure includes detecting the quantity of mucous, red blood cells, neutrophils and epithelial cells in the cytological material.

91. A method as claimed in claim 90, in which the purification procedure includes adding one or more of the following agents to the first receptacle in response to the quantity of mucous, red blood cells, neutrophils and epithelial cells detected: a mucous withdrawing agent, a red blood cell removing agent, and a neutrophils removing agent.

92. A method as claimed in claim 90, in which the purification procedure includes adding diluents to the first receptacle through the fluid inlet for reducing the viscosity of fluids therewithin.

93. A method as claimed in claim 89, in which the purification procedure includes draining supernatant from the first receptacle through the fluid outlet to leave epithelial cells in the first receptacle.

94. A method as claimed in claim 93, further comprising the step of transferring residual epithelial cells from the first receptacle to the second receptacle through the filter.

95. A method as claimed in claim 94, further comprising the step of transferring the second receptacle to a smearing unit, in which a portion of the epithelial cells from the second receptacle are ejected onto a specimen slide, and smeared on the slide.

96. A method as claimed in claim 95, in which a horizontal extent of the smeared epithelial cells is adjusted depending on the cellularity of the sample.

97. A method as claimed in claim 95, further comprising the step of fixing said smeared deposit on the slide using a chemical fixing agent, an air dryer, or other fixing means.

98. A method as claimed in claim 97, further comprising the step of staining the fixed epithelial cells using a Papanicolaou staining technique or a non-Papanicolaou staining technique.

99. A method as claimed in claim 82, in which the apparatus comprises: receiving means for receiving at least one deposit of a cytological specimen, first and second staining stations, and transfer means for transferring a portion of the or each deposit from the receiving means to the first staining station and/or a portion of the or each deposit to the second staining station, the first staining station being operable for a Papanicolaou staining technique and the second staining station being operable for a Papanicolaou staining technique and at least one further staining technique.

100. A method as claimed in claim 99, in which the receiving means comprises a tubular carousel having a plurality of reception ports extending along a peripheral longitudinal extent, each reception port being configured for receiving and holding a receptacle containing a deposit of a cytological specimen.

101. An method as claimed in claim 100, in which the carousel is configured to move in first or second modes, wherein in the first mode the carousel rotates about a central axis of rotation, and in the second mode, the carousel reciprocates about the axis of rotation.
